(19) United States
(12) Patent Application Publication
(10) Pub. No.: US 2025/0186352 A1
(43) Pub. Date: Jun. 12, 2025

(54) COMPRESSED PRODUCT COMPRISING PROBIOTICS AND METHOD FOR ITS PRODUCTION

(71) Applicant: BLUESTONE PHARMA GMBH, Baar (CH)

(72) Inventors: Danijela GAVRILOVIC, Leskovac (RS); Milan DMITROVIC, Belgrade (RS); Michael FABER, Sitges (ES); Karsten BRANDT, Sitges (ES)

(21) Appl. No.: 18/856,473

(22) PCT Filed: Apr. 13, 2023
(86) PCT No.: PCT/EP2023/059635

§ 371 Date: Oct. 11, 2024

(30) Foreign Application Priority Data
Apr. 13, 2022 (EP) 22168218.0

(51) Int. Cl. A61K 9/20; A61K 31/593; A61K 35/741

(52) U.S. Cl. CPC A61K 9/2095; A61K 9/2009; A61K 9/2013; A61K 9/2018; A61K 31/593; A61K 35/741

(57) ABSTRACT

Provided is a method for producing a compressed product, in particular a tablet, which comprises probiotic bacteria as active ingredient. In particular, a method is provided which comprises a briquetting step in which the excipients are compressed to large briquettes and fractioned to the desired particle size before the probiotic bacteria are added for final compression.

### FIELD OF THE INVENTION

The invention relates to a method for producing compressed products, in particular tablets, which comprise probiotic bacteria as active ingredient. In particular, the method comprises a briquetting step in which the excipients are compressed to large briquettes and fractioned to the desired particle size before the probiotic bacteria are added for final compression. Furthermore, the invention is directed to the compressed product produced by said method.

### BACKGROUND OF THE INVENTION

Probiotics are defined as live microorganisms which when administered in adequate amounts confer a health benefit on the host. The beneficial effects of probiotics may be mediated by a direct antagonistic effect against specific groups of undesired organisms, resulting in a decrease of their numbers, by an effect on the metabolism of such groups of organisms or by a general stimulatory effect on the immune system of animal or human hosts.

Probiotic microorganisms have been identified among microorganisms classified as yeasts, fungi and bacteria. For instance, lactic acid bacteria are in general recognized as being useful as probiotics or “probiotically active” organisms, i.e., organisms that may beneficially affect animal or human hosts.

Compressed tablets are the dominant dosage form for self-administration of pharmaceutical compositions. Tablets can be produced at a high speed and consequently at low prices and consumers are used to and frequently prefer this dosage form. To provide health benefits, the suggested minimal for bacteria is concentration probiotic 10⁶ CFU/g of a product (Shah (2000), J Dairy Sci 83 (4): 894-907). Thus, there is a constant need for methods which allow the manufacture of compressed tablets containing viable probiotically active organisms.

### SUMMARY OF THE INVENTION

The present invention relates to the surprising finding that probiotically active microorganisms can be formulated into a compressed product using a method which comprises a briquetting step in which the excipients are compressed to large briquettes, fractioned to the desired particle size and grinded with a granulator, and which further comprises a step wherein the probiotics are mixed with these granules, i.e. the grinded briquettes, and wherein this mixture is compressed to the final product. An overview of this method is depicted in FIG. 1.

In the method of the present invention, all excipients including the lubricant, but excluding the active pharmaceutical ingredients (APIs), here the probiotics and optionally a further active ingredient, are pressed to briquettes in a first step. Afterwards, fractionizing and grinding is performed and the APIs are mixed to the grinded briquettes, wherein this mixture is used for final tablet pressing. Thus, this method substantially differs from the commonly known dry granulation method, which involves compaction of powder particles into large pieces or compacts which are subsequently broken down into granules to produce granules that can be further processed into dosage forms, wherein before dry granulation, the formulation has to be completed including both the APIs and the excipients, minus the lubrication.

The method of the present invention surprisingly results in that the viability of the cells during tablet manufacturing only decreased from 7.2E+09 CFU/g prior to compression to 6.7E+9 CFU/g of the freshly prepared tablet composition; see Example 1. This corresponds to a decrease in viability of the cells of only about 7% during tablet pressing. In comparison, when applying a corresponding method without that briquetting step, i.e., wherein the excipients and the APIs, here the probiotics and optionally vitamin D3, are mixed and directly pressed to the final product, the viability of the cells during tablet manufacturing decreased from 7.33E+09 CFU/g prior to compression to 2.1E+9 CFU/g of the freshly prepared tablet composition; see Example 1. This corresponds to a decrease in viability of the cells of about 70% during tablet pressing. Thus, the method of the present invention is highly advantageous over a “non-briquetting” method, i.e. a method using direct compression. The comparative “non-briquetting” method is illustrated in FIG. 2. Furthermore, the compressed product prepared by the method of the present invention is less hard (75 N) than the corresponding product produced by the “non-briquetting” method (90 N), but remarkably has a lower friability, thus it is more stable than the product prepared by the “non-briquetting” method, i.e. 1.28% vs. 1.78%; see Example 1. The water activity and the disintegration time of both products is similar as can be derived from Table 3 in Example 1.

The method of the present invention applies only a compression force of 4.8 kN/compressed product, i.e. about 4.8 kN/cm², wherein the “non-briquetting” method requires a higher compression force, i.e. 5.2 kN/compressed product in order to produce the final product. Without intending to be bound by theory, the higher survival rate of the probiotics might be due to the lower compression force used in the method of the present invention since it is known that microorganisms are sensitive to the pressure used for tablet pressing.

Thus, the present invention relates to a method of producing a compressed product comprising probiotic bacteria as active ingredient and at least one or more excipients, wherein the method comprises at least mixing and homogenizing, i.e. blending, the excipients, which is followed by briquetting the excipients. Afterwards, the briquettes are fractionized and grinded and the probiotic bacteria are added to the grinded briquettes, followed by mixing and blending. Finally, the mixture is compressed to the compressed product. To guarantee a low water activity of the compressed product, the excipients are usually dried before compression and the presence of external water should be avoided. Furthermore, during tablet pressing, the generation of excessive heat should be avoided and pressing is performed with a pressure which allows the compaction of the mixture but without being lethal for the probiotic organisms.

In a preferred embodiment, the compressed product produced with the method of the present invention is a tablet and more preferably a lozenge or a chewable tablet. The compressed product preferably comprises lactic acid bacteria which are in general recognized as being useful as probiotics or “probiotically active” organisms, i.e. organisms that may beneficially affect animal or human hosts. The compressed product comprises in a preferred embodiment bacteria of the genus Streptococcus salivarius, preferably strain K12, ENT-K12, 24SMB, Rosell®-83, or HA-188, but most preferably strain K12 or ENT-K12, Lactobacillus rhamnosus, preferably strain LGG, Lactobacillus casei, preferably strain 431, Lactobacillus paracasei, preferably strain LP-33 (also designated as GMNL-32 or GM-080) or strain GMNL-133, Lactobacillus fermentum, preferably strain LC40, Lactobacillus crispatus, preferably strain M247, Bifidobacterium animalis subsp. lactis, preferably strain BB-12, Lactobacillus plantarum, preferably strain 299v, Lactococcus lactis, Enterococcus faecalis, Lactobacillus reuteri protectis, or a mixture of any one thereof, preferably of two of the mentioned strains.

In principle, any excipients can be used in the method of the present invention which are suitable for direct compression and/or dry granulation. In a preferred embodiment, the method of the present invention comprises the use of fructose and maltodextrin as bulking agents, silicon dioxide, or rice hulls as natural substitute for silicon dioxide as anti-caking agent, magnesium stearate, tricalcium phosphate, a rice extract blend, or a oat fiber blend as natural substitute for magnesium stearate, as lubricant, and optionally an aromatizing agent like strawberry, mint, orange, banana, passionfruit, cocoa, menthol, yuzu, lemon flavor, and/or combinations thereof, preferably passionfruit, cocoa and menthol; yuzu and mint; orange and mint flavor.

The present invention also relates to the compressed product, in particular to a tablet, which is produced by the method of the present invention as well as to a corresponding container, like a bottle or blister pack.

As it is commonly known in the art and shown in several (clinical) studies, probiotic organisms have a health benefitting effect and can even be used for the treatment of various diseases; see for example Wescombe et al., Future Microbiol. (2012) 7 (12), 1355-1371; Zupancic et al., Probiotics Antimicrob Proteins (2017) 9 (2), 102-110; Wilcox et al., Clin Microbiol Infect (2019) 25 (6), 673-680; Marom et al., Medicine (Baltimore) (2016) 95 (6), e2695; Clark, Curr Opin Immunol (2020) 66, 42-49; Bertuccioli et al., Nutrafoods (2019) 2, 80-88. Accordingly, the present invention relates to the compressed product produced by the method of the present invention for use in the treatment of otitis, preferably otitis media; upper respiratory tract infections, preferably tonsillitis or pharyngitis; lower respiratory tract infections, preferably bronchitis or pneumonia; diseases and inflammations of the oral cavity, preferably oral mucositis, candidiasis, and/or oral lichen planus; halitosis; skin disorders, preferably acne and/or dermatitis; gastro-intestinal problems; allergies or immune diseases preferably allergic rhinitis, or mastitis in a subject.

Furthermore, the present invention relates to the non-medical use of the compressed product for supporting healthy mouth microflora, healthy upper and lower respiratory tract microflora, healthy skin, improving weight management, maintaining healthy gut microflora, healthy vaginal flora; maintaining a normal digestion or supporting healthy gut mobility, bowel movement and/or healthy stool frequency, stool consistency and/or form in a subject.

Further embodiments of the present invention will be apparent from the description, the Figures and Examples that follow.

### BRIEF DESCRIPTION OF THE DRAWINGS

FIG. 1: Manufacturing flowsheet for a tableting process comprising a briquetting step.

FIG. 2: Manufacturing flowsheet for a tableting process without a briquetting step.

### DETAILED DESCRIPTION OF THE INVENTION

The present invention relates to a method of producing a compressed product. In particular, the inventors found a generally useful method by which a compressed product, preferably a tablet, can be produced which comprises viable, probiotically active organisms. This method comprises a briquetting step in which the excipients are compressed to a large briquette which is fractioned to the desired particle size and grinded with a granulator. Afterwards, the probiotics are mixed with the granules, i.e. the grinded briquettes, wherein this mixture is used for compression of the final product.

Thus, the method of the present invention comprises mixing and blending the excipients. Blending is performed to achieve blend uniformity and to distribute the lubricant. The optimum mixing time and speed to achieve a homogenous mixture can be evaluated by the person skilled in the art. For mixing and blending a fluidized bed granulator is preferably used. This fluid bed granulation is not only used for mixing and blending, but also for drying the excipients.

After mixing and blending of the excipients, a briquetting step is followed, i.e. the excipients are formed to briquettes, also referred to as large (flat) tablets, pallets, flakes, large pieces or compacts. In principle, this step can be performed by various methods, for example by a slugging technique or roller compaction. Slugging involves compression of primary powder particles into large flat tablets or pallets using a tablet press or a large heavy-duty rotary press, wherein roller compaction (also referred to as ribbon blending) is a process where formulation ingredients are continuously passed between two counter-rotating rollers where it is densified and consolidated into a sheet of solid mass. Depending on the type of rollers used, the feed material may be compacted into dense ribbon-like materials known as flakes (smooth rolls) or dense briquettes (almond or stick-shaped) if the rollers have grooved or etched surfaces. In the method of the present invention, the compression of the excipients into the briquettes is preferably performed by compression with a tablet press.

In this step, the compression force is not that critical since the probiotically active organisms, which are usually sensitive to pressure caused when formed into tablets, are not yet present in the formulation. Accordingly, a compression force has to be chose which is sufficient for compaction of the excipients. Such a compression force can be evaluated by a person skilled in the art. The compression force can be chosen between 1 to 50 kN/compressed product or/cm², respectively, preferably between 1 to 40 kN/compressed product or/cm², respectively, preferably between 1 to 30 kN/compressed product or/cm², respectively, preferably between 1 to 20 kN/compressed product or/cm², respectively, more preferably between 1 to 10 kN/compressed product or/cm², respectively and most preferably around 7 to 8 kN/compressed product/cm², respectively, in particular 7.5 kN/compressed product or/cm², respectively.

The next step comprises the fractionizing of the briquettes and grinding. Fractionizing is preferably performed in an oscillatory mill and grinding is preferably performed in a fluid bed granulator. Furthermore, a sieving step is usually performed ensuring a homogenous particle size. In the next step, the APIs are added to the fluid bed granulator. In particular, the probiotics are added and optionally a further active ingredient. In a preferred embodiment, a substantially pure composition of the probiotic is added. Thus, the probiotics which are added to the grinded briquettes are not mixed before with further excipients. Thus, the probiotics are added as substantially pure composition.

Again mixing and homogenizing, i.e. blending is performed. Preferably, segment mixing is performed, i.e, wherein the probiotic bacteria, and optionally the further active ingredient are mixed stepwise with parts of the excipient, in particular are mixed with a small part of the excipients, preferably with 10% of the excipients, then with a bigger part of the excipients, preferably further 40% of the excipients, and afterwards with the remaining part of the excipients, preferably 50% of the excipients. In a preferred embodiment, mixing is performed for about 25 minutes at 20 o/min.

Afterwards, the mixture is compressed to the final product by using a tablet press. Here, the compression force is a critical parameter since many probiotically active organisms, including the most interesting probiotic lactic bacteria, are highly sensitive to the pressure caused when formed into tablets by direct compression. Thus, a compression force has to be chosen which allows survival of the probiotics but which leads to a tablet with the desired properties, i.e. the properties as described below regarding water activity, disintegration time, average mass, resistance to crushing, CFU count and/or friability.

As a general rule: the lower the compression force, the higher the survival rate. However, a low compression force results in less coherent tablets, but with the method of the present invention, tablets with a good coherency and a high survival rate of the probiotic organisms have been produced as for example compared to tablets which have been produced with a method which does not comprise the “briquetting step; see Example 1. The compression force can be chosen in principle as described above for the generation of the briquettes but is preferably lower. Preferably the compression force is between 1 and 10 kN/compressed product or/cm², respectively, preferably between 2 and 9 kN/compressed product or/cm², preferably between 3 and 8 kN/compressed product or/cm², preferably between 3 and 7 kN/compressed product or/cm², preferably between 3 and 6 kN/compressed product or/cm², preferably between 4 and 6 kN/compressed product or/cm², more preferably around 5 kN/compressed product or/cm², respectively and in particular 4.8 kN/compressed product or/cm², respectively.

Also the dwell-time, i.e. the time during which the compression is maximal, needs to be considered. According to a preferred embodiment the compression time, i.e. the short period of time wherein the compressible formulation is compressed in the tablet press, is less than 1 s, preferably the time is less than 0.5 s, more preferably less than 0.1 s and most preferably about 0.08 s.

Another point is the heat development during compression. Since probiotics are heat sensitive, the generation of excessive heat during compression should be avoided. Thus, the tablet press is preferably run at a low speed in order to avoid strong heat development. The optimal speed can be tested by a person skilled in the art but preferably the tablet press is run at such speed that about 600 tablets are pressed per minute.

After compression, the compressed product is optionally analyzed regarding its disintegration time, its average mass, its resistance to crushing, its friability, its appearance, and/or its water activity. This can be performed by methods known in the art and in particular by the methods as described in the Examples section.

Before compression, the excipients which are used in the method of the present invention are preferably weighted and sieved, preferably through a 1 mm net, and furthermore dried in the fluid bed granulator which is also used for mixing and homogenizing the excipients as described above.

The probiotic microorganisms which are used in the method of the present invention can be provided in several forms, but preferably they are provided freeze-dried. Those are also weighted and sieved, preferably through a 1 mm net before they are added to the granules, i.e. to the grinded briquettes. Optionally, they are thermostabilized for about 1 to 3 hours before the weighting and sieving step. In case a compressed product is produced which comprises further active ingredients, the active ingredient(s) is (are) also weighted before adding to and mixing with the granulated excipients.

In order to achieve a low water activity of the compressed product which is important for its shelf-life, most of the steps, in particular the production steps, are performed at room temperature and at a relative humidity of about ≤35%. Room temperature can be defined as a temperature of ≤25° C. and more preferably of 15° C. to 25° C. Preferably at least the thermostabilizing step, as well as the steps of sieving the excipients, weighting and sieving the probiotic bacteria, and compressing the mixture to the compressed product are performed at said temperature and humidity. If the further active ingredient as defined above is present, weighting is also performed at the indicated temperature and humidity.

In one embodiment, the method of the present invention relates to the production of a compressed product which is a tablet. Tablets are solid dosage forms usually obtained by single or multiple compression of powders or granules. They can be either coated or uncoated. Tablets are normally right circular solid cylinders, the end surfaces of which are flat or convex and the edges of which may be beveled. They may have lines or break-marks (scoring), symbols, or other markings. Tablets are single-dose preparations intended for oral administration. Some are intended to be swallowed whole, some after being chewed and some after being crushed, some are intended to be dissolved or dispersed in water before being taken and some are intended to be retained in the mouth where the active ingredient(s) is/are liberated.

The different categories of tablets include uncoated tablets; coated tablets (including film-coated and sugar-coated tablets); soluble tablets; dispersible tablets; effervescent tablets; chewable tablets; tablets for use in the mouth (including sublingual and buccal tablets); and modified-release tablets (including delayed-release tablets (gastro-resistant/enteric-coated tablets) and sustained-release tablets (extended-/prolonged-release tablets)).

Tablets for use in the mouth like chewable tablets and lozenges, most preferably lozenges are a favored delivery form since especially children associate a lozenge with a candy and since sucking a tablet is generally more convenient than swallowing a whole tablet leading to a high acceptance of such a product. Thus, in a preferred embodiment, the method of the present invention is used for the production of compressed product which is a tablet for use in the mouth, preferably a chewable tablet or a lozenge, most preferably a lozenge.

Chewable tablets are usually uncoated and are intended to be chewed before being swallowed. Tablets for use in the mouth are also usually uncoated and they are usually formulated to effect a slow release and local action of the active ingredient(s) (for example, compressed lozenges) or the release and absorption of the active ingredient(s) under the tongue (sublingual tablets) or in other parts of the mouth (buccal) for systemic action.

A favorable feature of the compressed product produced by the method of the present invention is its long disintegration time and due to this the compressed product remains for a long time in the oral cavity during sucking before it dissolves. This is particular useful when probiotic bacteria are comprised in the compressed product which are useful in the treatment of upper and lower respiratory tract infections, diseases and inflammations of the oral cavity, and halitosis or which are used for supporting healthy mouth microflora, healthy upper and lower respiratory tract microflora since the longer sucking time allows the probiotic bacteria to efficiently colonize the oral cavity and the respiratory tract.

However, even when the compressed product comprises probiotic bacteria which rather act in the gastro-intestinal tract, lozenges are still a preferred way of administration since it rather reminds on a candy than on a medicament or health stimulating agent. Furthermore, for a fast majority of people, sucking a tablet is more convenient than swallowing a tablet.

Thus, in one embodiment, the compressed product produced by the method of the present invention has a disintegration time of more than 5 minutes, preferably between 10 and 30 minutes, more preferably around 15 minutes.

Furthermore, as can be derived from the Examples, the method of the present invention results in that the viability of the cells during tablet manufacturing only decreased from 7.2E+09 CFU/g prior to compression to 6.7E+9 CFU/g directly after compression, i.e., in the freshly prepared tablet composition; see Example 1. This corresponds to a decrease in viability of the cells of only about 7% during tablet pressing. In comparison, when applying a corresponding method without that briquetting step, i.e., wherein the excipients and the APIs, here the probiotics and optionally the further active ingredient, are mixed and directly pressed to the final product, the viability of the cells during tablet manufacturing decreased from 7.33E+09 CFU/g prior to compression to 2.1E+9 CFU/g of the freshly prepared tablet composition; see Example 1. This corresponds to a decrease in viability of the cells of about 70% during tablet pressing. Thus, the method of the present invention is highly useful for producing compressed products comprising probiotically active microorganisms since the method of the present invention has a beneficial effect on the survival of the probiotically active microorganisms.

Thus, in one embodiment, the viability of the cells during compression in a tablet press does not decrease by more than 10%, preferably by no more than 8%, more preferably by no more than 7%. The decrease in viability of the cells is calculated as: ([the total number of viable cells present in 1 g of the mixture prior to compression]−[the total number of cells present in 1 g of the freshly prepared tablet composition]) divided by [the total number of viable cells present in 1 g of the mixture prior to compression], multiplied by [100%].

Furthermore, the compressed product prepared by the method of the present invention has a hardness (resistance to crushing) of about 75 N, which is less hard than the corresponding product produced by the “non-briquetting” method (90 N), but remarkably has a lower friability. Thus, it is more stable than the product prepared by the “non-briquetting” method, i.e. 1.28% vs. 1.78%. Tablet hardness is the force (load) required to break a tablet and friability describes the tendency of a solid substance to break into smaller pieces under duress or contact.

The water activity of a compressed product is important for its shelf-life and the water activity of the compressed product as produced by the method of the present invention directly after compression is between about 0.1 and 0.2, preferably about 0.1.

Accordingly, the compressed product produced by the method of the present invention has a disintegration time of about 15 minutes, a hardness of about 75 N, a friability of about 1.3%, a water activity of about 0.1, and/or comprises probiotically active microorganisms in an amount of about 6.7E+9 CFU/g.

The International Scientific Association for Probiotics and Prebiotics defines “probiotics” as “live microorganisms that, when administered in adequate amounts, confer a health benefit on the host”. These microorganisms, which consist mainly of bacteria but also include yeasts, are naturally present in fermented foods, may be added to other food products, and are available as dietary supplements, like the compressed product of the present invention.

In principle any probiotic strain can be formulated in the compressed product. Health benefits have mainly been demonstrated for specific probiotic strains of the following genera: Aerococcus, Bacillus, Bacteroides, Bifidobacterium, Clostridium, Enterococcus, Fusobactehum, Lactobacillus, Lactococcus, Leuconostoc, Melissococcus, Micrococcus, Pediococcus, Peptostrepococcus, Propionibacterium, Staphylococcus, Streptococcus and Weissella. Thus, the compressed product preferably comprises species of any one of those genera or mixtures thereof and those are added to and mixed with the grinded briquettes in the method of the present invention, respectively.

Common microorganisms which may be formulated in the compressed product include but are not limited to the following:
Lactic acid bacteria: Genus Lactobacilli spp.; Species: Lactobacillus acidophilus, L. alimentarius, L. amylovorus, L. brevis, L. bulgaricus, L. casei, L. cellobiosus, L. crispatus, L. curvatus, L. delbrueckii spp. bulgaris, L. delbrueckii spp. lactis, L. farciminus, L. fermentum, L. gallinarum, L. helveticus, L. johnsonii, L. lactis, L. paracasei, L. plantarum, L. reuteri, L. rhamnosus, L. sake, L. salivarius; Genus: Streptococcus spp. Species: Streptococcus salivaris spp., S. salivarius (for example K12 or ENT-K12), S. faecalis, S. faecium; Genus: Lactococcus ssp., Species: L. lactis cremoris, L. lactis; Genus: Leuconostoc, Species: Lc. mesenteroides; and Genus: Pediococcus spp., Species: P. pentosaceus, P. acidilactici.
Bifidobacteria: Genus: Bifidobacterium spp., Species: B. adolescentis, B. animalis, B. bifidum, B. breve, B. essensis, B. infantis, B. laterosporum, B. thermophilum, B. longum.
Propionibacteria: Genus: Propionibacterium spp., Species: P. acidipropionici, P. freudenreichii, P. jensenii, P. thoenii.
Enterobacteria: Genus: Enterococcus spp., Species: E. fecalis, E. faecium. E. durans.
Sporulated bacteria: Genus: Bacillus spp., Species: B. alcolophilus, B. cereus, B. clausii, B. coagulans, B. subtilis.
Other bacteria: Genus: Escherichia coli, Species: E. coli; Genus: Sporolactobacillus spp. Species: S. imulinis.
Yeasts: Genus: Saccharomyces spp., Species: S. cerevisae (boulardii); that isolated from litchi fruit in Indonesia have also been accepted and used as probiotics.

In a preferred embodiment, the compressed product as produced by the method of the present invention comprises lactic acid bacteria or bifidobacteria, preferably those mentioned above and those are added to and mixed with the grinded briquettes in the method of the present invention, respectively. As used herein the term “lactic acid bacteria” refers to gram-positive, microaerophilic or anaerobic bacteria which ferment sugars with the production of acids including lactic acid as the predominantly produced acid, acetic acid, formic acid and propionic acid. Lactic acid bacteria are particularly preferred.

In a particular preferred embodiment, the probiotic bacteria are selected from the group consisting of: Streptococcus salivarius, preferably strain K12 (DSM 13084; BAA-1024), ENT-K12 (DSM 34540), 24SMB (DSM 23307), Rosell®-83, or HA-188, but most preferably strain K12 and ENT-K12, respectively, Lactobacillus rhamnosus, preferably strain LGG (ATCC 53103; US 4,839,28), Lactobacillus casei, preferably strain 431 (ATCC 55544), Lactobacillus paracasei, preferably strain LP-33 (also designated as GMNL-32 or GM-080; CCTCC M 204012, U.S. Pat. No. 6,994,848 B2) or strain GMNL-133 (CCTCC M 2011331, EP 2 581 461 B1), Lactobacillus fermentum, preferably strain LC40 (CECT5716, U.S. Pat. No. 7,468,270 B2), Lactobacillus crispatus, preferably strain M247 (LMG P-23257, EP 1 930 018 A1), Bifidobacterium animalis subsp. Lactis, preferably strain BB-12 (DSM 15954, EP 2 990 045 B1), Lactobacillus plantarum, preferably strain 299v (DSM 9843), Lactococcus lactis (Zamfir et al. 2016 Int J Food Sci Technol, 51, 2164-2170), Enterococcus faecalis (DSM 16440), Lactobacillus reuteri protectis, (DSM 17938) and/or any combinations thereof.

An accession number starting with “DSM” indicates that the strain is deposited with the Deutsche Sammlung von Mikroorganismen Und Zellkulturen GmbH (DSMZ), Mascheroder Weg lb, D-38124 Braunschweig, GERMANY; a number starting with “ATCC” or “BAA” indicates that the strain is deposited with the American Type Culture Collection (ATCC), P.O. Box 1549, Manassas, VA 20108, USA; a number starting with “CCTCC” indicates that the strain is deposited with the China Center for Type Culture Collection (CCTCC), College of Life Sciences, Wuhan University Wuhan 430072, China; a number starting with “CETC” indicates that the strain is deposited with the Colección Española de Cultivos Tipo (CECT), Edificio 3 CUE, Parc Cientific Universitat de Valencia, Catedrático Augustin Escardino 9, 46980 Paterna (Valencia); a number starting with “LMG” indicates that the strain is deposited with the BCCM/LMG Bacteria Collection, Laboratorium voor Microbiologie, Ghent University, K. L. Ledeganckstraat 35, B-9000 Ghent, Belgium.

In one preferred embodiment, the strain is S. salivarius K12, which is publicly available at the American Type Culture Collection (ATCC), P.O. Box 1549, Manassas, VA 20108, USA under Accession No. BAA-1024.

In one preferred embodiment, the strain is S. salivarius ENT-K12, which is genetically identical to S. salivarius K12 and which has been deposited at the Leibniz-Institut DSMZ-Deutsche Sammlung von Mikroorganismen und Zellkulturen GmbH, Inhoffenstraße 7B, 38124 Braunschweig, Germany, and assigned Accession Number DSM 34540 (Date of deposit: Feb. 22, 2023). The depositor is the Probionet GmbH, Schützenstrasse 380, 9100 Herisau, Switzerland.

The invention is not, however, limited to these above-mentioned particular microorganisms. The person skilled in the art would recognize those microorganisms, which may be useful in the method according to the invention. A very important embodiment of the present invention is to combine two or more of the above mentioned probiotically active organisms, such as e.g. a preparation comprising a probiotically active Lactobacillus species and a probiotically active Streptococcus species.

The compressed product produced by the method of the present invention preferably comprises the following probiotically active organisms, i.e. those are used in the method of the present invention:
Streptococcus salivarius, preferably K12 or ENT-K12
Lactobacillus rhamnosus, preferably strain LGG
Lactobacillus casei, preferably strain 431
Lactobacillus paracasei, preferably strain LP-33 and/or GMNL-133
Lactobacillus fermentum, preferably strain LC40
Lactobacillus crispatus, preferably strain M247
Lactobacillus plantarum, preferably strain 299v
Bifidobacterium animalis subsp. lactis, preferably strain BB-12
Lactococcus lactis
Streptococcus salivarius and Lactobacillus rhamnosus, preferably Streptococcus salivarius K12 or ENT-K12, and Lactobacillus rhamnosus strain LGG;
Streptococcus salivarius and Lactobacillus casei, preferably Streptococcus salivarius K12 or ENT-K12, and Lactobacillus casei 431;
Streptococcus salivarius and Lactobacillus fermentum, preferably Streptococcus salivarius K12 or ENT-K12, and Lactobacillus fermentum strain LC40;
Streptococcus salivarius and Lactobacillus crispatus, preferably Streptococcus salivarius K12 or ENT-K12, and Lactobacillus crispatus strain M247;
Streptococcus salivarius and Lactobacillus plantarum, preferably Streptococcus salivarius K12 or ENT-K12, and Lactobacillus plantarum strain 299v;
Streptococcus salivarius and Bifidobacterium animalis subsp. lactis, preferably Streptococcus salivarius K12 or ENT-K12, and Bifidobacterium animalis subsp. lactis strain BB-12;
Streptococcus salivarius and Lactococcus lactis, preferably Streptococcus salivarius K12 or ENT-K12, and Lactococcus lactis;
Streptococcus salivarius and Lactobacillus paracasei, preferably Streptococcus salivarius K12 or ENT-K12, and Lactobacillus paracasei LP-33;
Streptococcus salivarius and Lactobacillus paracasei, preferably Streptococcus salivarius K12 or ENT-K12, and Lactobacillus paracasei GMNL-133.

In one embodiment, any of the above-mentioned combinations which include a Streptococcus salivarius strain include Streptococcus salivarius K12.

In one embodiment, any of the above-mentioned combinations which include a Streptococcus salivarius strain include Streptococcus salivarius ENT-K12.

Of course, combinations any other of the above-mentioned strains and/or of three, four or more of the above-mentioned strains are also possible and encompassed.

The formulation for producing the compressed product with the method of the present invention comprises one or more excipients and optionally one or more further active ingredients. The further active ingredients can be compounds that support and/or boost the immune system like vitamins and minerals. Those include, but are not limited to vitamin A, vitamin D3, vitamin B6, vitamin C, folic acid, vitamin B12, iron, zinc, copper, selenium and combinations thereof. In a preferred embodiment, the further active ingredient is vitamin D3 (cholecalciferol), for example in a form of a premix.

Excipients are helpful and/or necessary to prepare solid dosage forms. They are widely involved in the flow properties of mixtures, compression properties in tablet preparation, disintegration properties for smooth disintegration, sticking properties in which powder sticks to the surface of tablet compression punches to cause defects on the tablet surface, capping in which the upper portion of tablets is peeled off in a cap shape during compression, lamination in which a tablet is peeled off in a layered fashion, and binding properties for enhancing the hardness of tablets.

Excipients include bulking agents, diluents, carrier, binders, lubricants, disintegrators, anti-caking agents, preservatives, colors or flavors. The excipients are preferably pharmaceutically acceptable and/or acceptable for human consumption.

A carrier or bulking agent means a vehicle for delivery of the probiotic microorganism, to the individual, in which the vehicle is compatible with cell viability, or activity of the extract. Acceptable carrier/bulking agents suitable for use in the administration of viable probiotic microorganisms are well known to those skilled in the art; see, for example, Remington's Pharmaceutical Sciences, 18th ed., Gennaro, ed., 1990, Mack Publishing Co., Easton, Pa., incorporated herein by reference. Preferably, the carrier/bulking agent is a pharmaceutically acceptable carrier/bulking agent and/or a carrier/bulking agent acceptable for human consumption. Acceptable carriers/bulking agents suitable for use with probiotically active microorganisms in the compressed product of the present invention are usually solid carriers known in the art and include, but are not limited to magnesium carbonate; magnesium stearate; celluloses; talc; sugars such as fructose, sucrose, mannitol, sorbitol, xylitol, lactose; sugar substitutes such as isomalt; starches; maltodextrin; flours; (fructose-) oligosaccharides and skim milk, and similar edible powders, but are not limited thereto.

Typical diluents, by way of example, are starches; lactose; mannitol; kaolin; calcium phosphate or sulphate; inorganic salts such as sodium chloride; and powdered sugars and sugar substitutes as mentioned above or celluloses.

Typical binders include starch; gelatin; sugars such as lactose, fructose, and glucose; and the like. Natural and synthetic gums are also convenient, including acacia; alginates; locust bean gum; methylcellulose, e.g., Hydroxypropyl methylcellulose (HMPC); polyvinylpyrrolidine (PVP) tragacanth; PVP K-30; PVP K-25; xanthan gum; and the like. Polyethylene glycol (PEG 4000 or PEG 6000); ethyl cellulose; and waxes can also serve as binders as well as Nu-BIND® and CompactCel®DIS.

Lubricants and anti-caking agents to prevent sticking during formulation and to prevent the formation of lumps include slippery solids such as talc, silica, magnesium and calcium stearate, polyethylene glycol, stearic acid, hydrogenated vegetable oils, rice extract blend, for example Nu-MAG®, CompactCel®LUB, in particular CompactCel® F clear 290.02 LUB, oat fiber blend, for example CompactCel® F 200.28 LUB, potato starch, gum Arabica, CompactCel®FLO, Nu-FLOW®, silicon dioxide, tricalcium phosphate, and rice hulls, for example Nu-FLOW®. or CompactCel®FLO.

Disintegrators are substances which swell when wetted to break up the composition and release the S. salivarius or extract. The disintegrators include starches; clays; celluloses; algins and gums; more particularly corn and potato starches; methylcellulose; agar; bentonite; wood cellulose; cation exchange resins; alginic acid; guar gum; citrus pulp; carboxymethylcellulose; powdered sponge; silica; and sodium lauryl sulfate.

Aromatizing agents are known to the person skilled in the art and can be of any kind which give a good (or at least a different taste) to the compressed product. Those flavors include, but are not limited to strawberry, mint, orange, banana, passionfruit, cocoa, menthol, yuzu, lemon and/or combinations thereof, preferably passionfruit, cocoa and menthol; yuzu and mint; orange and mint.

In a preferred embodiment, the excipients used in the method of the present invention for producing the compressed product comprise one or more bulking agent(s) like sugar or sugar substitute, a lubricant, an anti-caking agent and optionally a flavor.

In recent years, as consumer interest in well-being and natural foods has increased, the use of natural additives in place of synthetic additives in the food industry has gradually increased. In particular, in pharmaceutical drugs and health functional foods, safety and environmentally friendly factors in production processes are considered important, and thus many products containing natural components are being developed and the market size thereof is also increasing.

Accordingly, in a preferred embodiment, the formulation for producing the compressed product with the method of the present invention does comprise as few additives as possible and thus, only further comprises (next to the probiotic microorganisms and the bulking agent(s)) a lubricant and an anti-caking agent as excipient, but no other excipients like disintegrators. However, an aromatizing agent, preferably a natural flavor is optionally comprised in the excipients used in the method of the present invention. In one embodiment, the bulking agent as used in the method of the present invention is a sugar or a sugar substitute, like fructose, maltodextrin, isomalt, or isomaltulose, or a mixture thereof. Preferably, the compressed product as produced by the method of the present invention comprises maltodextrin and fructose as bulking agents, i.e., the bulking agents as used in the method of the present invention are preferably maltodextrin and fructose.

In one embodiment, the lubricant as used in the method of the present invention is magnesium stearate, or a natural replacement. In one embodiment, the anti-caking agent as used in the method of the present invention is silicon dioxide, tricalcium phosphate, or a natural replacement. Natural lubricants are known in the art and are for example a crude fat-containing bean powder as described in WO 2013/165131 A1, a rice extract blend, for example the product Nu-MAG®, or the product CompactCel® LUB (CompactCel® F clear 290.02 LUB, Biogrund GmbH, Huenstetten, Germany), an oat fiber blend, for example the product CompactCel® LUB (CompactCel® F 200.28 LUB, Biogrund GmbH, Huenstetten, Germany) or further products, like potato starch, or gum Arabica. Natural anti-caking agents are also known in the art and are for example powdered cellulose, like JELUCEL®, native potato starch, inulin, rice fibers, rice hulls, for example Nu-FLOW®, or CompactCel® FLO.

Thus, in one embodiment, magnesium stearate and/or silicon dioxide, and/or tricalcium phosphate is (are) substituted by a rice extract blend, like Nu-MAG® or CompactCel® LUB, potato starch, gum Arabica, rice hulls, like CompactCel® FLO, or Nu-FLOW® in the method of the present invention.

In a preferred embodiment, APIs used in the method of the present invention are any one of the probiotic bacteria or combinations of probiotic bacteria listed above, and the excipients are fructose, maltodextrin, magnesium stearate, silicon dioxide and a flavor preferably selected from those mentioned above, most preferably strawberry flavor. Alternatively, magnesium stearate is substituted with a natural lubricant, preferably with an oat fiber blend, preferably comprising oat fibers, microcrystalline cellulose and sunflower oil refined, for example the product CompactCel® F 200.28 LUB, or with a rice extract blend, preferably comprising rice extract, microcrystalline cellulose, and sunflower oil refined, for example the product CompactCel® F clear 290.02 LUB, or any one of Nu-MAG®, potato starch, gum Arabica, or, but most preferably with a rice extract blend; and silicon dioxide is substituted with tricalcium phosphate, or a natural anti-caking agent, preferably rice hulls, for example the product Nu-FLOW®, or CompactCel®FLO.

Accordingly, in one embodiment, the product, in particular the compressed product of the present invention comprises any one of the probiotic bacteria or combinations of probiotic bacteria listed above, as well as fructose, maltodextrin, a rice extract blend, preferably comprising rice extract, rice hulls, gum arabic and sunflower oil, for example the product Nu-MAG®, silicon dioxide, and the above-mentioned flavor.

In one embodiment, the product, in particular the compressed product of the present invention comprises any one of the probiotic bacteria or combinations of probiotic bacteria listed above, as well as fructose, maltodextrin, a rice extract blend, preferably comprising rice extract, microcrystalline cellulose and sunflower oil refined, for example the product CompactCel®LUB, silicon dioxide, and the above-mentioned flavor.

In one embodiment, the product, in particular the compressed product of the present invention comprises any one of the probiotic bacteria or combinations of probiotic bacteria listed above, as well as fructose, maltodextrin, an oat fiber blend, preferably comprising oat fiber, microcrystalline cellulose and sunflower oil refined, for example the product CompactCel®LUB, silicon dioxide, and the above-mentioned flavor.

In one embodiment, the product, in particular the compressed product of the present invention comprises any one of the probiotic bacteria or combinations of probiotic bacteria listed above, as well as fructose, maltodextrin, magnesium stearate, rice hulls, preferably Nu-FLOW®, and the above-mentioned flavor.

In one embodiment, the product, in particular the compressed product of the present invention comprises any one of the probiotic bacteria or combinations of probiotic bacteria listed above, as well as fructose, maltodextrin, magnesium stearate, rice hulls, preferably CompactCel®FLO, and the above-mentioned flavor.

In one embodiment, the product, in particular the compressed product of the present invention comprises any one of the probiotic bacteria or combinations of probiotic bacteria listed above, as well as fructose, maltodextrin, a rice extract blend, preferably comprising rice extract, rice hulls, gum arabic and sunflower oil, for example the product Nu-MAG®, rice hulls, preferably Nu-FLOW®, and the above-mentioned flavor.

In one embodiment, the product, in particular the compressed product of the present invention comprises any one of the probiotic bacteria or combinations of probiotic bacteria listed above, as well as fructose, maltodextrin, a rice extract blend, preferably comprising rice extract, microcrystalline cellulose and sunflower oil refined, for example the product CompactCel®LUB, rice hulls, preferably Nu-FLOW®, and the above-mentioned flavor.

In one embodiment, the product, in particular the compressed product of the present invention comprises any one of the probiotic bacteria or combinations of probiotic bacteria listed above, as well as fructose, maltodextrin, an oat fiber blend, preferably comprising oat fiber, microcrystalline cellulose and sunflower oil refined, for example the product CompactCel®LUB, rice hulls, preferably Nu-FLOW®, and the above-mentioned flavor.

In one embodiment, the product, in particular the compressed product of the present invention comprises any one of the probiotic bacteria or combinations of probiotic bacteria listed above, as well as fructose, maltodextrin, a rice extract blend, preferably comprising rice extract, rice hulls, gum arabic and sunflower oil, for example the product Nu-MAG®, rice hulls, preferably CompactCel®FLO, and the above-mentioned flavor.

In one embodiment, the product, in particular the compressed product of the present invention comprises any one of the probiotic bacteria or combinations of probiotic bacteria listed above, as well as fructose, maltodextrin, a rice extract blend, preferably comprising rice extract, microcrystalline cellulose and sunflower oil refined, for example the product CompactCel®LUB, rice hulls, preferably CompactCel®FLO, and the above-mentioned flavor.

In one embodiment, the product, in particular the compressed product of the present invention comprises any one of the probiotic bacteria or combinations of probiotic bacteria listed above, as well as fructose, maltodextrin, an oat fiber blend, preferably comprising oat fiber, microcrystalline cellulose and sunflower oil refined, for example the product CompactCel®LUB, rice hulls, preferably CompactCel®FLO, and the above-mentioned flavor

Nu-MAG®, Nu-FLOW®, NuBind®, CompactCel®LUB, CompactCel®FLO, and CompactCel®DIS are natural excipients, i.e., natural lubricants, anti-caking agents, and binders, respectively. They are composed of natural organic ingredients and do not comprise for example silica or magnesium stearate and are thus suitable for “clean label” products. For example, Nu-MAG® is a rice extract blend and comprises rice extract, rice hulls, gum arabic and sunflower oil. Nu-FLOW® is a rice concentrate (a concentrate of silica from rice) and comprises rice hulls. NuBind® is a gum fiber blend and comprises guar gum, gum arabic, agave fiber, rice hulls, and agave syrup, CompactCel®LUB is available in two variants, i.e., either comprising rice extract, microcrystalline cellulose and sunflower oil refined, or oat fiber, microcrystalline cellulose and sunflower oil refined.

Optionally, one or more further active ingredients can be used, which are for example those vitamins and minerals mentioned above. In a preferred embodiment, vitamin D3 is used as further active ingredient in the method of the present invention. Vitamin D3 can be formulated into the compressed product in form of a vitamin D3 premix, which comprises 2.5 μg/mg vitamin D3. In one embodiment, the compressed product of the present invention comprises 5 to 10 μg vitamin D3, i.e. cholecalciferol.

The compressed products produced by the method of the present invention weigh at least enough to formulate 5 mg of the probiotically active organisms, preferably to formulate at least 30 mg of the probiotic microorganism and only weights so much that it is still convenient to suck the compressed product. The weight of the compressed product can be from about 100 mg to about 2000 mg, preferably from about 500 mg to about 1000 mg. Preferably, the compressed product weighs about 950 mg±10 mg.

The amounts of the excipient(s) and active ingredient(s) may vary and a person skilled in the art knows how to properly mix the tablet ingredients in order to produce a compressed product which has the desired properties, i.e. the properties as described above regarding water activity, disintegration time, average mass, resistance to crushing, and/or friability.

The excipients comprise in total at least 10% and up to 99.5% of the final formulation which is used in the method of the present invention, preferably about 87% to 95%, more preferably about 87%, 89%, 93%, 94%, or 95%. In this context, fructose comprises about 40% to 80%, preferably about 50% to 70%, more preferably about 60% of the formulation, silicon dioxide and magnesium stearate as well as their natural substitutes each comprise about 1% to 2%, preferably around 1.6% of the formulation and the flavor comprises about 0.5 to 2%, preferably about 1% of the formulation. Optionally the further active ingredient, like vitamin D3 is present and in this case the formulation comprises 5-10 μg vitamin D3 (cholecalciferol), which can be provided for example in a form of a premix (either about 0.2% or 0.4%). The vitamin D3 premix comprises 2.5 μg pure vitamin D3 per mg premix.

Maltodextrin comprises about 10% to 50%, preferably about 15% to 40%, more preferably about 21% to 34% and most preferably between 29.6% and 30%, between 28.5% and 28.9%, between 27.5% and 27.9%, between 23.3% 23.7%, or between 15.9% and 16.3% of the formulation.

The probiotic microorganisms comprise at least 0.5% and up to 20% of the formulation. Preferably the content of the probiotic microorganisms is between 3% and 13%, more preferably about 4.2%, 5.3%, 6.3%, 10.5% or 12.6%. Assuming that the compressed product weights 950 mg, it comprises between 5 mg to 160 mg probiotic bacteria, preferably between 30 mg and 120 mg, more preferably 40 mg, 50 mg, 60 mg, 100 mg or 120 mg.

For example, the compressed product produced by the method of the present invention comprises 100 mg or 120 mg of Lactobacillus paracasei in total, either 100 mg or 120 mg of each of the strains LP-33 or GMNL-133 alone or 100 mg or 120 mg of both strains in combination; or 50 mg of Streptococcus salivarius K12 and ENT-K12, respectively; or 30 mg of Streptococcus salivarius K12 and ENT-K12, respectively, and 30 mg of Lactobacillus rhamnosus LGG; or 30 mg of Streptococcus salivarius K12 and ENT-K12, respectively, and 30 mg of Lactobacillus casei 431; or 30 mg of Streptococcus salivarius K12 and ENT-K12, respectively, and 30 mg of Bifidobacterium animalis subsp. lactis BB-12; or 20 mg of Streptococcus salivarius K12 and ENT-K12, respectively, 20 mg of Lactobacillus rhamnosus LGG and 20 mg of Bifidobacterium animalis subsp. lactis BB-12; or 20 mg of Streptococcus salivarius K12, 20 mg of Lactobacillus casei and 20 mg of Bifidobacterium animalis subsp. lactis BB-12; or 50 mg of Streptococcus salivarius K12 and ENT-K12, respectively, and 50 mg of Lactobacillus paracasei LP-33; or 50 mg of Streptococcus salivarius K12 and ENT-K12, respectively, and 50 mg of Lactobacillus paracasei GMNL-133.

It will be appreciated that useful probiotically active organisms can be of a genetically modified strain of one of the above organisms. The term “genetically modified” as used herein indicates any modification of DNA sequences coding for genes and modifications of sequences that regulate the expression of genes. Accordingly, genetic modification can be based on construction or selection of mutants of microorganism or it can be based on recombinant DNA technology.

As used herein the term “mutant” is comprised by the conventional meaning of that term, i.e. it refers to strains obtained by subjecting a microbial strain to any conventionally used mutagenization treatment including treatment with a chemical mutagen such as ethanemethane sulphonate (EMS) or N-methyl-N′-nitro-N-nitroguanidine (NTG), UV light or to spontaneously occurring mutants which are selected on the basis of a desired characteristic such as e.g. antibiotic and/or gastric acid-resistance. It is also possible to select useful genetically modified organisms according to the invention by random mutagenesis or by selection of spontaneously occurring mutants, i.e. without the use of recombinant DNA technology. Mutants of the above-mentioned organisms also can be provided by recombinant DNA technology including site-directed mutagenesis, PCR techniques and other in vitro or in vivo modifications and insertion of DNA sequences.

The compressed product produced with the method of the present invention preferably comprises 10⁵, 10⁶, 10⁹ or even 10¹¹ CFU/g. Preferably, the compressed product comprises between 5×10⁹ and 10×10⁹ CFUs of the probiotic microorganisms. This will be obtained by using about 40 mg to 120 mg of the probiotic strains per compressed product when producing the compressed product of the present invention.

An exemplarily compressed product as produced with the method of the present invention comprises the ingredients as shown in Table 2:

TABLE 2
Ingredients of an exemplarity probiotics tablet produced
with the method of the present invention.

| | | Amount | Percentage |
|---|---|---|---|
| | Function | (mg) | (%) |
| Streptococcus salivarius | Active ingredient/probiotic | 50.0 | 5.26 |
| K12/ENT-K12 | microorganism | | |
| Fructose | Bulking agent | 585 | 61.58 |
| Maltodextrin | Bulking agent | 275 | 28.95 |
| Silicon dioxide | Anti-caking agent | 15 | 1.58 |
| Magnesium Stearate (vegetable) | Lubricant | 15 | 1.58 |
| Strawberry flavor | Flavor | 10 | 1.05 |
| Total | | 950 | 100 |

In case more of the probiotic microorganisms are comprised in the compressed product, the amount of maltodextrin will be reduced so that the compressed product still weights about 950 mg. In case less of the probiotic microorganisms are comprised in the compressed product, the amount of maltodextrin will be increased so that the compressed product still weights about 950 mg. Optionally, the compressed product comprises 5-10 μg of a vitamin and/or mineral, for example vitamin D3 and in this case 2 or 4 mg of a vitamin D3 premix and in this case, the amount of maltodextrin will be adapted accordingly.

Of course, as also mentioned above, the anti-caking agent and/or the lubricant can be replaced by its corresponding natural substitutes.

The present invention further relates to a package comprising the compressed product obtainable by the method of the present invention, for example a container, i.e. bottle or a blister pack.

As mentioned above, probiotics are defined as live microorganisms that, when administered in adequate amounts, confer a health benefit on the host. The beneficial effects of probiotics may be mediated by a direct antagonistic effect against specific groups of undesired organisms, resulting in a decrease of their numbers, by an effect on the metabolism of such groups of organisms or by a general stimulatory effect on the immune system of animal or human hosts. Thus, the present invention relates to the use of the compressed product obtainable by the method of the present invention for reducing the occurrence of infections with pathogens for example in the oral cavity, the respiratory tract, the gastrointestinal tract and the vagina or of the skin. Accordingly, the compressed product is used for supporting healthy mouth microflora, healthy upper and lower respiratory tract microflora, healthy skin, improving weight management, maintaining healthy gut microflora, healthy vaginal flora; maintaining a normal digestion or supporting healthy gut mobility, bowel movement and/or healthy stool frequency, stool consistency and/or form in a subject.

In the present context, the expression “reducing the occurrence of infections” indicates that the above-mentioned infections or symptoms caused by the presence of pathogens occurs at a reduced frequency or seriousness as compared to a human or animal subject who or which is not being treated with the compressed product. In the present context treatment is also to be construed as encompassing prevention or prophylaxis in addition to cure.

The invention also relates to a method for reducing the occurrence of infections with pathogens for example in the oral cavity, the respiratory tract, the gastrointestinal tract and the vagina or of the skin, wherein the method comprises administering the compressed product according to the invention to a subject, preferably wherein the compressed product is a tablet, preferably a chewable tablet or lozenge and is administered orally. Accordingly, the invention relates to a method for supporting healthy mouth microflora, healthy upper and lower respiratory tract microflora, healthy skin, improving weight management, maintaining healthy gut microflora, healthy vaginal flora; maintaining a normal digestion or supporting healthy gut mobility, bowel movement and/or healthy stool frequency, stool consistency and/or form by administering the compressed product according to the invention to a subject.

It is furthermore documented that probiotic microorganisms produce essential vitamins and nutrients required by the intestinal cells and furthermore assist with degradation of certain nutrients and even activate cell-mediated immune effector functions. Thus probiotic microorganisms can improve the general health status of a mammal.

A number of reports indicate that intake of probiotic microorganisms may not only reduce the occurrence of infections and are health promoting, but indeed contribute to the treatment of disease; see for example Wescombe et al., Future Microbiol. (2012) 7 (12), 1355-1371; Zupancic et al., Probiotics Antimicrob Proteins (2017) 9 (2), 102-110; Wilcox et al., Clin Microbiol Infect (2019) 25 (6), 673-680; Marom et al., Medicine (Baltimore) (2016) 95 (6), e2695; Clark, Curr Opin Immunol (2020) 66, 42-49; Bertuccioli et al., Nutrafoods (2019) 2, 80-88. Thus, the present invention relates to a compressed product for use in the treatment of various diseases or conditions like otitis, preferably otitis media; upper respiratory tract infections, preferably tonsillitis or pharyngitis; lower respiratory tract infections, preferably bronchitis or pneumonia; diseases and inflammations of the oral cavity, preferably oral mucositis, candidiasis, and/or oral lichen planus; halitosis; skin disorders, preferably acne and/or dermatitis; gastro-intestinal problems like diarrhea, gastroenteritis, irritable bowel syndrome (IBS), inflammatory bowel disease (IBD), urogenital infection; allergies, lactose intolerance or immune diseases preferably allergic rhinitis, or mastitis in a subject.

These health benefitting effects have been verified is various studies and described for example in Fijan (2014), Int J Environ Res Public Health. 11 (5): 4745-4767; EP 1 483 366 B1; EP 2 581 461 B1; U.S. Pat. No. 6,994,848 B2; WO 2005/007178 A1; Di Pierro et al., Drug Healthc Patient Saf. 6 (2014), 15-20; Wescombe et al., Future Microbiol. (2012) 7 (12), 1355-1371; Zupancic et al., Probiotics Antimicrob Proteins (2017) 9 (2), 102-110; Wilcox et al., Clin Microbiol Infect (2019) 25 (6), 673-680; Marom et al., Medicine (Baltimore) (2016) 95 (6), e2695; Clark, Curr Opin Immunol (2020) 66, 42-49; Bertuccioli et al., Nutrafoods (2019) 2, 80-88.

The invention also relates to a method for treatment of the above-mentioned conditions/diseases in a subject, wherein the method comprises administering the compressed product according to the invention to the subject.

The term “individual” or “subject” as used herein includes humans, horses, dogs, cats, pigs, sheep, cattle, goats but is not limited thereto. Preferably, the individual is a human. The compressed product of the present invention can be administered to the individual at any age, e.g., childhood, adolescence, or adulthood.

In general, the amount of probiotics administered via the compressed product of the present invention to the individual will be an amount of an active agent high enough to deliver the desired benefit, but low enough to avoid serious side effects. Specific dosages can vary widely according to various individual variables including size, weight, age, disease severity and responsiveness to therapy. Methods for determining the appropriate dosage may be determined by the consumer as they deem appropriate, or on a case-by-case basis by an attending a pharmacist or clinician. Such determinations are routine to one of ordinary skill in the art (see for example, Remington's Pharmaceutical Sciences, 8th ed., Gennaro, ed., Mack Publishing Company, Easton, Pa., 1990).

The compressed product of the present invention may need to be administered to the patient once only or more usually repeatedly. Repeat prophylactic or therapeutic treatments may be once a month, once a week, once a day, twice a day, three times a day, four times a day, five times a day, six times a day or as may otherwise be required.

Furthermore, the present invention relates to the compressed product as obtained with the method of the present invention, preferably which has the characteristics as mentioned above, i.e. the water activity, disintegration time, average mass, resistance to crushing, CFU count and/or friability as defined above and shown in the Examples.

The compressed product comprises the ingredients, i.e. the excipients and APIs (in an amount) as described with regard to the method of the present invention, above.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

A more complete understanding can be obtained by reference to the following specific Examples which are provided herein for purposes of illustration only and are not intended to limit the scope of the invention.

### EXAMPLES

### Material and Methods

Method of the Present Invention, i.e. Production of a Compressed with a Briquetting Step

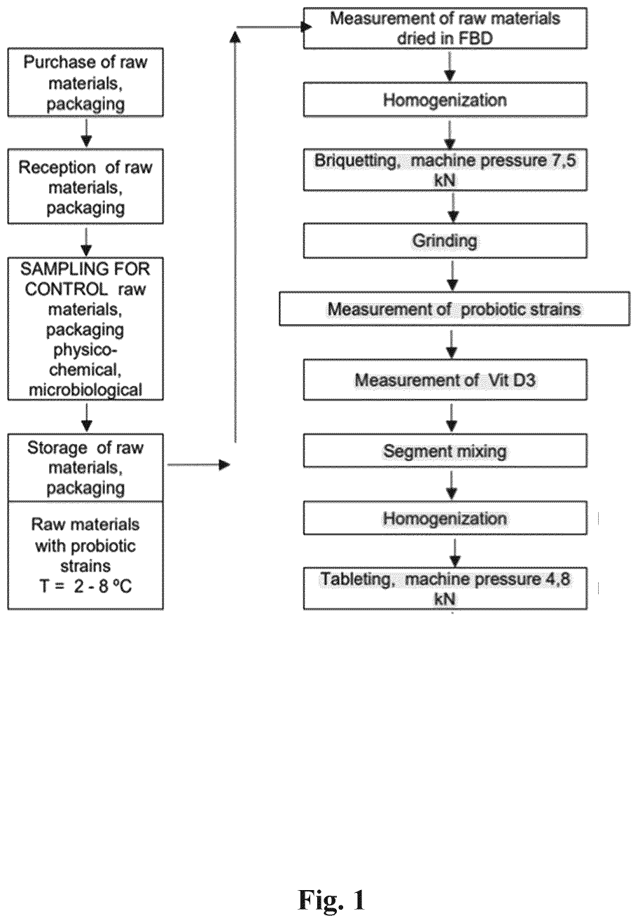

The method is performed according to the schema depicted in FIG. 1 and is exemplarily described for the production of tablets comprising probiotic microorganisms, fructose, maltodextrin, magnesium stearate, silicon dioxide, strawberry flavor, and vitamin D3. However, the same process is used for tablets which are defined hereinbefore.

The method comprises a briquetting step in which the excipients are compressed to large briquettes, fractioned to the desired particle size and grinded with a granulator, and further comprises a step wherein the probiotics are mixed with these granules, i.e. the grinded briquettes, and wherein this mixture is compressed to the final product.

In particular, in a first step, the excipients (here: fructose, maltodextrin, magnesium stearate, silicon dioxide strawberry flavor) are dried in a fluid bed granulator, weighted, sieved through a 1 mm net, mixed und homogenized, i.e. blended. Mixing and homogenizing is also performed in the fluid bed granulator.

Afterwards, briquetting is performed, wherein the homogenized mixture of excipients is pressed to a briquette using a table press. The compression force is about 7.5 kN. This is followed by fractionizing and grinding the briquettes.

Meanwhile the probiotic microorganisms, which are used as freeze-dried product, are allowed to thermostate for 3 h at 25° C. Afterwards, the probiotics are weighted and sieved through a 1 mm net, and vitamin D3 is weighted as well.

In a next step, 10% of the grinded briquettes, i.e. the excipients, are mixed with the probiotic microorganisms and vitamin D3. Afterwards, further 40% of the grinded briquettes are added and mixed and finally the remaining 50% of the grinded briquettes are added and mixed. Mixing is performed for about 25 minutes at 20 o/min (segment mixing). The next step comprises homogenizing, i.e. blending and final tablet pressing. Tablet pressing is performed at about 4.8 kN and the tablet press is run at low speed in order to avoid strong heat production, for example at a speed to produce 600 tablets/min. The pressed tablets are analyzed with regard to their appearance, average mass, resistance to crushing, disintegration time, friability and/or water activity. All tablet producing steps are performed at room temperature, in particular at <25° C., and at a relative humidity of <35%.

The concentration of the excipients and the active pharmaceutical ingredients in this exemplarily process are as follows:
5.26% probiotics, here for example Streptococcus salivarius K12,
61.58% fructose
28.53% maltodextrin,
1.58% magnesium stearate,
1.58% silicon dioxide
1.05% strawberry flavor, and
0.42% vitamin D3 premix.

The vitamin D3 premix can be substituted with maltodextrin and if different amounts of the probiotic bacteria are used, the concentration of maltodextrin is adapted accordingly.

Comparative Method, i.e. Production of a Compressed Product without a Briquetting Step

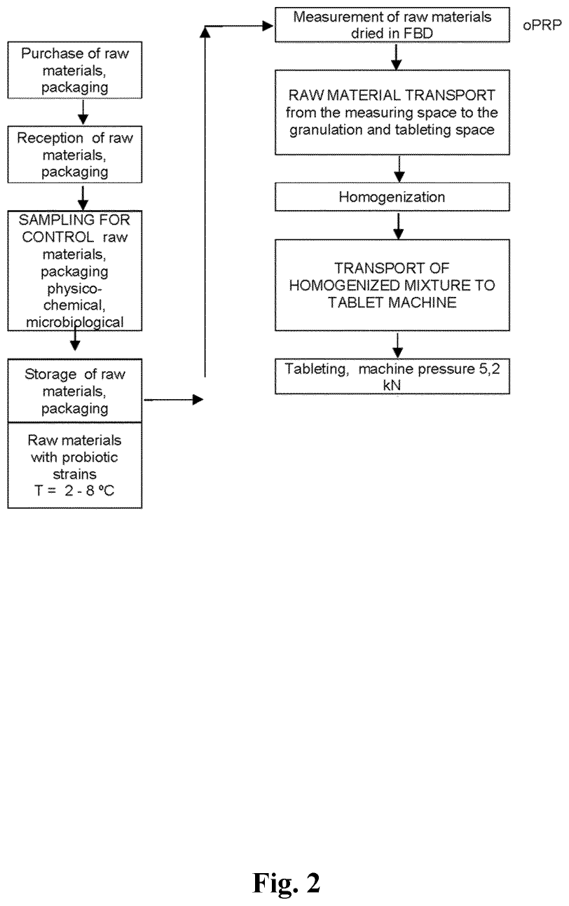

This method is performed according to the schema depicted in FIG. 2 and is exemplarily described for tablets comprising probiotic microorganisms, fructose, maltodextrin, magnesium stearate, silicon dioxide, strawberry flavor, and vitamin D3.

This method does not comprise a briquetting step, but all ingredients, i.e. excipients and active ingredients, here the probiotics and vitamin D3, are directly compressed to the final product.

In particular, in a first step, the excipients (here: fructose, maltodextrin, magnesium stearate, silicon dioxide strawberry flavor) are dried in a fluid be granulator, weighted, sieved through a 1 mm net and mixed. The sieving is performed at room temperature, in particular at <25° C., and at a relative humidity of <35%.

Meanwhile the probiotic microorganisms, which are used as freeze-dried product, are allowed to thermostate for 3 h at 25° C. Afterwards, the probiotics are weighted and sieved through a 1 mm net, and vitamin D3 is weighted as well, wherein those steps are performed at room temperature, in particular at <25° C., and at a relative humidity of <35%.

In a next step, 10% of the excipients are mixed with the probiotics and vitamin D3. Afterwards, further 40% of the excipients are added and mixed and finally the remaining 50% of the excipients are added and mixed. Mixing is performed for about 25 minutes at 20 o/min. The next step comprises blending and tablet pressing. Tablet pressing is performed at about 5.2 kN and the tablet press is run at low speed in order to avoid strong heat production, for example at a speed to produce 600 tablets/min. The tablet pressing step is also performed at room temperature, in particular at <25° C., and at a relative humidity of <35%. The pressed tablets are analyzed with regard to their appearance, average mass, resistance to crushing, disintegration time, friability and/or water activity.

The concentration of the excipients and the active pharmaceutical ingredients in this exemplarily process are as follows:
5.26% probiotics, here for example Streptococcus salivarius K12,
61.58% fructose
28.53% maltodextrin,
1.58% magnesium stearate,
1.58% silicon dioxide
1.05% strawberry flavor, and
0.42% vitamin D3 premix.

The vitamin D3 premix can be substituted with maltodextrin and if different amounts of the probiotic bacteria are used, the concentration of maltodextrin is adapted accordingly.

### Determination of Colony Forming Units (CFU)

Determination of CFUs of S. salivarius, L. casei and L. rhamnosis is performed as described in “Istituto Superiore di Sanità, Metodi microbiologici tradizionali e metodi molecolari per l'analisi degli integratori alimentari a base di o con, probiotici per uso umano, by Paolo Aureli, Alfonsina Fiore, Concetta Scalfaro, Giovanna Franciosa, 2008, ii, 63 p. Rapporti ISTISAN 08/36, ISSN 1123-3117”, in particular in chapters 21 and 24.

### Determination of the Hardness of the Compressed Product

The determination of the hardness of the compress product, i.e. its resistance to crushing, is performed as described in chapter 2.9.8 of the European Pharmacopoeia 6.0 by Council of Europe; 6th Edition; published on May 10, 2008; ISBN-10:9287160546; ISBN-13:978-9287160546.

### Determination of the Disintegration Time of the Compressed Product

The determination of the disintegration time of the compressed product is performed as described in chapter 2.9.1 of the European Pharmacopoeia 6.0 by Council of Europe; 6th Edition; published on May 10, 2008; ISBN-10:9287160546; ISBN-13:978-9287160546.

### Determination of the Water Activity of the Compressed Product

The determination of the water activity is performed using the HygroLab 3 of Rotronic AG Bassersdorf, Switzerland, and measurement is performed according to the user manual.

### Determination of the Friability of the Compressed Product

The determination of the friability of the compressed product is performed as described in chapter 2.9.7 of the European Pharmacopoeia 6.0 by Council of Europe; 6th Edition; published on May 10, 2008; ISBN-10:9287160546; ISBN-13:978-9287160546.

### Example 1: Comparison of Tablets Comprising Probiotics Produced with a Method Comprising a “Briquetting” Step with Those Produced with a Method without a “Briquetting” Step

In the following, the tablets which are produced with the method of the present invention, i.e. with a method which comprises the above described “briquetting step”, are compared to tablets which are produced with a method which does not comprise this “briquetting step”, but in which all ingredients, i.e. excipients and active ingredients, here the probiotics and vitamin D3, are directly compressed to the final product.

The comparison is exemplarily made with tablets comprising 5.26% Streptococcus salivarius K12, 61.58% fructose, 28.53% maltodextrin, 1.58% magnesium stearate, 1.58% silicon dioxide, 1.05% strawberry flavor, and 0.42% vitamin D3 premix. In particular, these tablets weight 950 mg and comprise 50 mg S. salivarius K12 and have been analyzed regarding their hardness, disintegration time, friability, water activity and CFUs of S. salivarius K12. The results are shown in Table 3.

TABLE 3
Characteristics of 950 mg tablets comprising 50 mg S. salivarius K12 (CFU prior
to compression: 1.74E+11) which have been produced either with the method of the
present invention (briquetting) or with the comparative method (no briquetting).
The analyses have been performed directly after compression of the final product.

| | Hardness | Disintegration | Friability | Water activity | Premix, | |
|---|---|---|---|---|---|---|
| | [Newton] | time [min] | [%] | [Aw] | CFU/g | CFU/g K12 |
| Briquetting | 75 | 15 | 1.28 | 0.1 | 7.20E+09 | 6.72E+09 |
| No Briquetting | 90 | 15 | 1.78 | 0.138 | 7.33E+09 | 2.21E+09 |

As can be derived from Table 3, the tablets produced with the “briquetting” method are less hard, have a lower friability and a lower water activity than the tablets produced with the method without the briquetting step. The disintegration time is the same, but remarkably, the viability of the cells during tablet manufacturing using the “briquetting” method only decreased from 1.74E+11 CFU/g prior to compression to 6.7E+9 CFU/g of the freshly prepared tablet composition, wherein the viability of the cells during tablet manufacturing using the method without briquetting decreased from 1.74E+11 CFU/g prior to compression to 2.1E+9 CFU/g of the freshly prepared tablet composition. Thus, the method of the present invention has a beneficial effect on the survival of the probiotically active microorganisms.

1. A method of producing a compressed product comprising probiotic microorganisms as active ingredient and at least one or more excipients, wherein the method comprises at least the following steps:
(i) briquetting the excipients, preferably with a pressure of about 7.5 kN/compressed product;
(ii) fractionizing and grinding the briquettes;
(iii) adding the probiotic microorganisms to the grinded briquettes and mixing;
(iv) compressing the mixture to the compressed product, preferably with a pressure of about 4.8 kN/compressed product; and optionally
(vii) analyzing the disintegration time, the average mass, the resistance to crushing, the appearance, the friability, and/or the water activity of the compressed product.

2. The method of claim 1, which further comprises the following steps:
(a) drying, weighting and sieving of the excipient(s) before step (i), preferably wherein the drying is performed in a fluid be granulator;
(b) mixing and homogenizing the excipient(s) after step (a) and before step (i);
(c) weighting and sieving of the probiotic microorganisms before step (iii), preferably wherein sieving is performed through a 1 mm net; and/or
(d) homogenizing the mixture after step (iii) and before step (iv).

3. The method of claim 1 or 2, wherein the compressed product comprises one or more further active ingredients, preferably vitamins and/or minerals, more preferably vitamin D3, which is weighted, added to and mixed with the grinded briquettes and the probiotic microorganisms.

4. The method of any one of claims 1 to 3, wherein the method is performed at room temperature, preferably wherein room temperature is ≤25° C. and more preferably between 15° C. and 25° C., and at a relative humidity of about ≤35%.

5. The method of any one of claims 1 to 4, wherein the compressed product is a tablet, preferably a lozenge or a chewable tablet.

6. The method of any one of claims 1 to 5, wherein the probiotic microorganisms are lactic acid bacteria, preferably of the genus Streptococcus, Lactobacillus, Lactococcus, Enterococcus, and/or Bifidobacterium.

7. The method of any one of claims 1 to 6, wherein the probiotic microorganisms are selected from the group consisting of Streptococcus salivarius, preferably Streptococcus salivarius K12 or Streptococcus salivarius ENT-K12; Lactobacillus rhamnosus, preferably Lactobacillus rhamnosus LGG; Lactobacillus casei; Lactobacillus paracasei; Lactobacillus fermentum; Lactobacillus crispatus; Lactobacillus plantarum; Bifidobacterium animalis subsp. Lactis; Lactococcus lactis; Enterococcus faecalis; Lactobacillus reuteri; either alone or in combination.

8. The method of any one of claims 1 to 7, wherein the one or more excipients are one or more bulking agents, lubricants, anti-caking agents, and optionally aromatizing agents.

9. The method of claim 8, wherein the bulking agents are fructose and maltodextrin, the lubricant is magnesium stearate or a natural substitute, the anti-caking agent is silicon dioxide or a natural substitute, and the aromatizing agent is a flavor, preferably strawberry flavor.

10. The method of to any one of claims 1 to 9, wherein the mixture of excipients constitutes about 87% to 96%, preferably about 87%, 89%, 93%, 94%, or 95% of the compressed product and/or the probiotic bacteria constitute about 3% to 13%, more preferably about 4.2%, 5.3%, 6.3%, 10.5% or 12.6% of the compressed product; and/or wherein the compressed product comprises between 5 mg to 160 mg probiotic bacteria, preferably between 30 mg and 120 mg, more preferably 40 mg, 50 mg, 60 mg, 100 mg or 120 mg.

11. The method of any one of claims 1 to 10, wherein the compressed product has a hardness of about 75 kN, a friability of about 1.3%, and/or a disintegration time of about 15 minutes.

12. A compressed product, preferably a tablet, more preferably a lozenge or a chewable tablet, obtainable by the method of any one of claims 1 to 11.

13. The compressed product of claim 12 for use in the treatment of otitis, preferably otitis media; upper respiratory tract infections, preferably tonsillitis, and/or pharyngitis; lower respiratory tract infections, preferably pneumonia; diseases and inflammations of the oral cavity, oral mucositis, candidiasis, oral lichen planus, halitosis; skin disorders, preferably acne and/or dermatitis; gastro-intestinal problems; allergies or immune diseases, preferably allergic rhinitis in a subject.

14. Use of the compressed product of claim 12 or 13 for supporting healthy mouth microflora, healthy upper and lower respiratory tract microflora, healthy skin, healthy vaginal microflora, improving weight management, maintaining healthy gut microflora, maintaining a normal digestion or supporting healthy gut mobility, bowel movement and/or healthy stool frequency, stool consistency and/or form in a subject.

15. A package comprising the compressed product of claim 12 or 13, or as defined in claim 14, preferably wherein the package is a blister pack or bottle.
